(19) United States
(12) Patent Application Publication
(10) Pub. No.: US 2018/0020999 A1
(43) Pub. Date: Jan. 25, 2018

(54) BONE ANALYZING DEVICE

(71) Applicant: Shimadzu Corporation, Kyoto (JP)

(72) Inventor: Junya YAMAMOTO, Kyoto (JP)

(73) Assignee: Shimadzu Corporation, Kyoto (JP)

(21) Appl. No.: 15/550,527

(22) PCT Filed: Feb. 12, 2016
(86) PCT No.: PCT/JP2016/054164

§ 371 Date: Aug. 11, 2017

(30) Foreign Application Priority Data
Feb. 13, 2015 (JP) 2015-026647

(51) Int. Cl. A61B 6/00; A61B 6/02

(52) U.S. Cl. CPC A61B 6/505; A61B 6/025; A61B 6/54; A61B 6/5205; A61B 6/5217; A61B 6/461

(57) ABSTRACT

A bone analysis apparatus capable of calculating a more highly reliable fracture risk evaluation value. A bone density of the invention is positioned for a partial description of a fracture risk. That is, in the invention, it is considered that an accurate fracture risk cannot be sufficiently obtained only by one of the bone density and a structural parameter although both the bone density and the structural parameter are important to know the fracture risk. According to the invention, the fracture risk is comprehensively evaluated on the basis of the structural parameter for evaluating a structure of a trabecular bone in addition to the bone density. With this configuration, since the fracture risk can be evaluated while also taking the structure of the trabecular bone into consideration, the fracture risk can be more accurately evaluated.

### TECHNICAL FIELD

The present invention relates to a bone analysis apparatus which calculates a fracture risk evaluation value indicating a strength of a bone, and more particularly, to a bone analysis apparatus which calculates a fracture risk evaluation value on the basis of a bone density.

### BACKGROUND ART

Osteoporosis is a disease in which a bone becomes fragile. When the osteoporosis progresses, the risk of fracture increases. In order to prevent the fracture caused by osteoporosis, it is effective to daily diagnose how fragile bones are and to take measures beforehand in consideration of the diagnosis result (for example, see Patent Document 1).

There is a risk of fracture as an index for determining how fragile bones are. The fracture risk is an index showing how easily a fracture occurs and can be understood as an index showing how much bones can endure physical stress. In order to appropriately diagnose the bone state, this fracture risk needs to be calculated with high accuracy.

As a method of calculating the fracture risk, a method of measuring a bone density is known. The bone density is an index indicating a bone filled state. Bones such as a femur involving with an exercise are made of various materials. Even in the femur that seems to be the same in appearance, there is a case in which the contents of mineral components (bone minerals) contained in the bone may be different. Such a mineral component is necessary for strengthening the bone. The bone density is a numerical value indicating the density of bone mineral. The bone density can be measured relatively easily by X-ray imaging. This is because mineral components are easily imaged in that X-rays do not easily penetrate mineral components.

In fact, the bone density is a concept different from the fracture risk. That is, the fracture risk indicating the strength of bones cannot be accurately measured without the fracture of bones. However, the fracture test of bones cannot be performed in actual. From this situation, an idea of using the bone density as an index indicating the fracture risk has been suggested. The bone density can be simply known differently from the fracture risk evaluation value. Thus, a conventional apparatus is configured to estimate the fracture risk evaluation value through the bone density. That is, according to the idea of the conventional apparatus, it is estimated that the bone becomes stronger as the bone density becomes higher and the strength of the bone is analyzed on the basis of the estimation. Thus, according to the conventional apparatus, it is assumed that the bone having the same bone density have the same fracture risk. When the bone densities of femures of different subjects are the same, it is considered that the femures of these subjects have the same fracture risk.

Further, structural parameters that show the characteristics of spongy tissues composed of trabecular bones can also be used to know a health condition of bone (see Patent Document 1). Such structural parameters are, for example, indexes indicating the compactness of the trabecular bone. The structural parameters are numerical values indicating the bone state and are also used for diagnosis. The health state of the bone can be also understood as an index showing how easily the fracture of the bone occurs. Based on this idea, the bones having the same structural parameters also have the same fracture risk.

### CITATION LIST

### Patent Document

Patent Document 1: JP-A-2013-027608

### SUMMARY OF THE INVENTION

### Technical Problem

However, the conventional apparatus has the following problems. That is, it is difficult to mention that the evaluation of the fracture risk based on the conventional configuration is always correct.

In a medical institution using the conventional fracture risk evaluation device, a realization has been obtained in which the risk of fracture is not the same even when the subject has the same bone density. Certainly, it is considered that the fracture risk is different depending on the environment of the subject. However, there is a doubt that the bone density does not necessarily mean the fracture risk. In this way, there is a limitation in reliability in the conventional apparatus that knows the fracture risk only based on the bone density. The same applies to the structural parameters which are obtained as numerical values of the characteristics of spongy tissues. Regarding this point, the inventors of the invention have found that the reliability of the fracture risk is degraded since both the bone density and the structure of the spongy bone are not taken into consideration for the evaluation of the fracture risk in the related art. That is, the new knowledge is that the evaluation of the fracture risk is affected since a gap inside the bone is not considered in the evaluation only using the bone density considered to be sufficient in the related art and the evaluation of the fracture risk is not sufficient since the content of mineral is not considered only in the structure of the spongy bone.

The invention has been made in view of such circumstances and an object of the invention is to provide a bone analysis apparatus calculating a fracture risk evaluation value on the basis of a bone density, more specifically, a bone analysis apparatus capable of calculating a highly reliable result.

### Solution to Problem

The invention has the following configuration in order to solve the above-described problems.

That is, a bone analysis apparatus according to the invention includes: fracture risk estimation means for calculating a fracture risk evaluation value indicating a risk of causing a fracture of a bone of a subject on the basis of a structural parameter expressed as numerical values of characteristics of a spongy structure composed of a trabecular bone and a bone density of the subject.

### Operation/Effect

In the bone analysis apparatus that calculates the fracture risk evaluation value on the basis of the bone density, a more highly reliable result can be obtained. That is, the bone density of the invention is positioned for a partial description of the fracture risk. That is, in the invention, it is considered that an accurate fracture risk cannot be sufficiently obtained only by the bone density although the bone density is important to know the fracture risk. The same applies to the structural parameter. That is, in the invention, it is considered that an accurate fracture risk cannot be sufficiently obtained only by the structural parameter although the structural parameter is important to know the fracture risk. According to the invention, the fracture risk is comprehensively evaluated on the basis of the structural parameter for evaluating a structure of a trabecular bone in addition to the bone density. With this configuration, since the fracture risk can be evaluated by considering both the contents of minerals of bones and the gaps inside the bones from two viewpoints of the bone density and the structure of the trabecular bone, the fracture risk can be more accurately evaluated.

Further, in the above-described bone analysis apparatus, the fracture risk estimation means may calculate the fracture risk evaluation value by using data showing a correlation among the fracture risk evaluation value, the bone density, and the structural parameter.

### Operation/Effect

The above-described configuration more specifically shows the bone analysis apparatus of the invention. When the fracture risk estimation means calculates the fracture risk evaluation value by using data showing a correlation among the fracture risk evaluation value, the bone density, and the structural parameter, the fracture risk evaluation value can be calculated by repeating the same evaluation method in the subjects.

Further, the above-described bone analysis apparatus may further include structural parameter calculation means for calculating the structural parameter on the basis of a tomosynthesis image of the subject.

### Operation/Effect

The above-described configuration more specifically shows the bone analysis apparatus of the invention. Since the structural parameter can be calculated on the basis of the image in which the trabecular bone is clearly captured when the structural parameter is calculated on the basis of the tomosynthesis image of the subject, the fracture risk can be evaluated more accurately.

Further, in the above-described bone analysis apparatus, the bone density may be acquired on the basis of an inspection different from the capturing of the tomosynthesis image.

### Operation/Effect

The above-described configuration more specifically shows the bone analysis apparatus of the invention. It is difficult to accurately calculate the bone density by the tomosynthesis image. Thus, since the bone density can be accurately calculated when the bone density is obtained by a dedicated imaging operation different from the imaging of the tomosynthesis image, the fracture risk can be evaluated more accurately.

Further, the above-described bone analysis apparatus may further include input means for inputting the bone density by an operator.

### Operation/Effect

The above-described configuration more specifically shows the bone analysis apparatus of the invention. When the operator includes the input means for inputting the bone density, the bone density obtained by an apparatus different from the bone analysis apparatus can be reliably input to the bone analysis apparatus.

Further, the above-described bone analysis apparatus may further include storage means for storing the bone density. The invention can be also applied to a configuration without the input means.

Further, in the above-described bone analysis apparatus, the structural parameter calculation means may calculate any one of a value BV/TV indicating a ratio between a bone component inside an interested region involving with the calculation of the structural parameter and the other part, a value TSL indicating a total extension of the trabecular bone, and a value TbTh indicating a width of the trabecular bone as the structural parameter.

### Operation/Effect

The above-described configuration shows a specific configuration of the bone analysis apparatus of the invention. When the structural parameter calculated by the structural parameter calculation means is any one of the value BV/TV, the value TSL, and the value TbTh, the bone analysis apparatus of the invention can be more reliably realized.

Further, the above-described bone analysis apparatus may further include: gray-level co-occurrence matrix generation means corresponding to the structural parameter calculation means and generating a gray-level co-occurrence matrix by counting the number of times of pixels separated from each other by a predetermined distance and appearing in an interested region as a combination of pixel values, a pair of two pixels having a combination of predetermined pixel values among pixels constituting the interested region involving with the calculation of the structural parameter; and texture analysis means for performing a texture analysis on the basis of the gray-level co-occurrence matrix and calculating a texture analysis index corresponding to the structural parameter as the structural parameter.

Further, one or more of correlation, dissimilarity, contrast, homogeneity, entropy, angular second moment, variance, and inverse differential moment may be selected as the texture analysis index calculated by the texture analysis means.

### Operation/Effect

The above-described configuration shows a specific configuration of the bone analysis apparatus of the invention. The above-described texture index value is an existing structural parameter and can be relatively easily calculated. Thus, according to the above-described configuration, the bone analysis apparatus of the invention can be more reliably realized.

Further, the above-described bone analysis apparatus may further include: a radiation source that irradiates a radiation; radiation source movement means for moving the radiation source relative to the subject; radiation source movement control means for controlling the radiation source movement means; detection means for detecting a radiation transmitted through the subject; detector movement means for moving the detection means relative to the subject; detector movement control means for controlling the detector movement means; image generation means for generating an image on the basis of an output of the detection means; and tomographic image generation means for generating the tomosynthesis image on the basis of a continuously shot image obtained while moving the radiation source and the detection means relative to the subject.

### Operation/Effect

The above-described configuration shows a specific configuration of the bone analysis apparatus of the invention. The invention can be also applied to the above-described digital tomosynthesis apparatus.

### Advantageous Effects of the Invention

According to the invention, in the bone analysis apparatus that calculates the fracture risk evaluation value on the basis of the bone density, a more highly reliable result can be obtained. That is, the bone density of the invention is positioned for a partial description of the fracture risk. That is, in the invention, it is considered that an accurate fracture risk cannot be sufficiently obtained only by the bone density although the bone density is important to know the fracture risk. The same applies to the structural parameter. That is, in the invention, it is considered that an accurate fracture risk cannot be sufficiently obtained only by the structural parameter although the structural parameter is important to know the fracture risk. According to the invention, the fracture risk is comprehensively evaluated on the basis of the structural parameter for evaluating a structure of a trabecular bone in addition to the bone density. With this configuration, since the fracture risk can be evaluated from two viewpoints of the bone density and the structure of the trabecular bone, the fracture risk can be more accurately evaluated.

### BRIEF DESCRIPTION OF THE DRAWINGS

FIG. 1 is a functional block diagram showing an overall configuration of a bone analysis apparatus according to a first embodiment.

FIG. 2 is a schematic diagram showing a tomosynthesis image capturing principle according to the first embodiment.

FIG. 3 is a functional block diagram showing a detail of an analysis unit according to the first embodiment.

FIG. 4 is a functional block diagram showing an example of an analysis unit according to the first embodiment.

FIG. 5 is a schematic diagram showing a concept of a fracture risk estimation unit according to the first embodiment.

FIG. 6 is a schematic diagram showing an operation of a trabecular shape analysis unit according to the first embodiment.

FIG. 7 is a schematic diagram showing an operation of a matrix generation unit according to the first embodiment.

FIG. 8 is a schematic diagram showing an operation of the matrix generation unit according to the first embodiment.

FIG. 9 is a schematic diagram showing an estimation expression according to the first embodiment.

FIG. 10 is a schematic diagram showing an estimation expression according to the first embodiment.

FIG. 11 is a schematic diagram showing the estimation expression according to the first embodiment.

FIG. 12 is a schematic diagram showing an effect of the first embodiment.

FIG. 13 is a schematic diagram showing an operation of a fracture risk estimation unit according to the first embodiment.

FIG. 14 is a schematic diagram showing an effect of a fracture risk evaluation according to the first embodiment.

FIG. 15 is a schematic diagram showing an effect of the fracture risk evaluation according to the first embodiment.

FIG. 16 is a schematic diagram showing a tomographic image capturing principle according to a second embodiment.

FIG. 17 is a schematic diagram showing the tomographic image capturing principle according to the second embodiment.

FIG. 18 is a schematic diagram showing the tomographic image capturing principle according to the second embodiment.

FIG. 19 is a schematic diagram showing the tomographic image capturing principle according to the second embodiment.

FIG. 20 is a schematic diagram showing a modified example of the invention.

### MODE FOR CARRYING OUT THE INVENTION

Hereinafter, embodiments of the invention will be described. An apparatus according to the invention is a bone analysis apparatus capable of evaluating a strength of a bone of a subject M. X-rays correspond to radiations of the invention and an FPD stands for a flat panel detector.

### First Embodiment

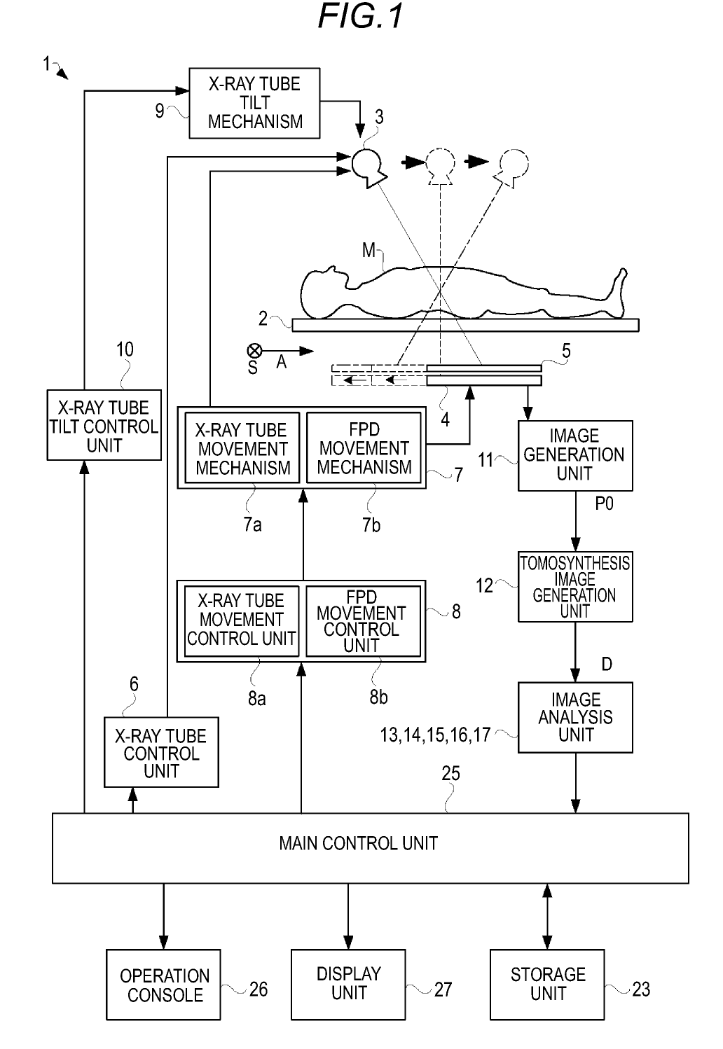

FIG. 1 is a functional block diagram showing a configuration of a bone analysis apparatus according to a first embodiment. As shown in FIG. 1, a bone analysis apparatus 1 according to the first embodiment includes a ceiling plate 2 which places a subject M corresponding to an X-ray tomography target thereon, an X-ray tube 3 which irradiates a cone-shaped X-ray beam to the subject M set on an upper portion (near one surface side of the ceiling plate 2) of the ceiling plate 2, an FPD 4 which is provided at a lower portion (near the other surface side of the ceiling plate) of the ceiling plate 2 and detects X-rays transmitted through the subject M, a synchronous movement mechanism 7 which synchronously moves the X-ray tube 3 and the FPD 4 in the opposite directions with an interested region of the subject M interposed therebetween while the center axis of the cone-shaped X-ray beam matches the center point of the FPD 4 at all times, asynchronous movement control unit 8 which controls the synchronous movement mechanism, and an X-ray grid 5 which is provided to cover an X-ray detection surface for detecting the X-rays of the FPD 4 and absorbs scattered X-rays . In this way, the ceiling plate 2 is disposed at a position interposed between the X-ray tube 3 and the FPD 4. The X-ray tube 3 corresponds to a radiation source of the invention and the FPD 4 corresponds to detection means of the invention.

The synchronous movement mechanism 7 includes an X-ray tube movement mechanism 7a which moves the X-ray tube 3 in a body axis direction A relative to the subject M and an FPD movement mechanism 7b which moves the FPD 4 in the body axis direction A relative to the subject M. Further, the synchronous movement control unit 8 includes an X-ray tube movement control unit 8a which controls the X-ray tube movement mechanism 7a and an FPD movement control unit 8b which controls the FPD movement mechanism 7b. The X-ray tube movement mechanism 7a corresponds to radiation source movement means of the invention and the FPD movement mechanism 7b corresponds to detector movement means of the invention. Further, the X-ray tube movement control unit 8a corresponds to radiation source movement control means of the invention and the FPD movement control unit 8b corresponds to detector movement control means of the invention.

The X-ray tube 3 is configured to repeatedly irradiate a cone-shaped pulsar X-ray beam to the subject Min accordance with the control of an X-ray tube control unit 6. A collimator for collimating the X-ray beam into a pyramid cone shape is attached to the X-ray tube 3. Then, the X-ray tube 3 and the FPD 4 respectively generate imaging systems 3 and 4 which capture X-ray transmission images.

The synchronous movement mechanism 7 is configured to synchronously move the X-ray tube 3 and the FPD 4. The synchronous movement mechanism 7 linearly moves the X-ray tube 3 along a linear track (a longitudinal direction of the ceiling plate 2) parallel to the body axis direction A of the subject M in accordance with the control of the synchronous movement control unit 8. The movement directions of the X-ray tube 3 and the FPD 4 match the longitudinal direction of the ceiling plate 2. Further, the cone-shaped X-ray beam which is irradiated from the X-ray tube 3 during an inspection is normally irradiated toward the interested part of the subject M and the X-ray irradiation angle is changed, for example, from an initial angle of −20° to a final angle of 20° in accordance with a change in the angle of the X-ray tube 3. Such a change of the X-ray irradiation angle is performed by an X-ray tube tilt mechanism 9. An X-ray tube tilt control unit 10 is provided for the purpose of controlling the X-ray tube tilt mechanism 9.

Then, the bone analysis apparatus 1 according to the first embodiment further includes a main control unit 25 which generally controls the control units 6, 8, and 10 and a display unit 27 which displays a tomosynthesis image D. The main control unit 25 is configured as a CPU and realizes the control units 6, 8, and 10 and units 11, 12, 13, 14, 15, 16, and 17 described below by performing various programs. The storage unit 23 stores all data of a trabecular analysis such as a control method of each unit and an estimation expression referred by the fracture risk estimation unit 17 to be described later. An operation console 26 is an input device which is used when an operator inputs the bone density to the bone analysis apparatus 1. The storage unit 23 corresponds to storage means of the invention and the operation console 26 corresponds to input means of the invention.

Further, the synchronous movement mechanism 7 linearly moves the FPD 4 provided at the lower portion of the ceiling plate 2 in the body axis direction A (the longitudinal direction of the ceiling plate 2) of the subject M in synchronization with the linear movement of the X-ray tube 3. Then, the movement direction is a direction opposite to the movement direction of the X-ray tube 3. That is, the focal position and the irradiation direction of the cone-shaped X-ray beam of the X-ray tube 3 change in accordance with the movement of the X-ray tube 3 and the X-ray beam is normally received by the entire surface of the X-ray detection surface of the FPD 4. In this way, the FPD 4 is configured to acquire, for example, seventy four perspective images PO while synchronously moving in a direction opposite to the X-ray tube 3 in one inspection. Specifically, the imaging systems 3 and 4 oppositely move to a position indicated by the one-dotted chain line shown in FIG. 1 through a position indicated by the broken line while a position indicated by the solid line is set as an initial position. That is, a plurality of X-ray transmission images are captured while the positions of the X-ray tube 3 and the FPD 4 are changed. Incidentally, since the cone-shaped X-ray beam is normally received by the entire surface of the X-ray detection surface of the FPD 4, the center axis of the cone-shaped X-ray beam always matches the center point of the FPD 4 during the imaging operation. Further, the center of the FPD 4 moves linearly during the imaging operation, but the direction of this movement is opposite to that of the movement of the X-ray tube 3. That is, the X-ray tube 3 and the FPD 4 are synchronously moved in the body axis direction A in the opposite directions. A symbol S shown in FIG. 1 indicates a body side direction of the subject M.

That is, the synchronous movement mechanism 7 performs an operation of moving the FPD 4 toward the other end side of the ceiling plate 2 in the longitudinal direction in synchronization with the movement of the X-ray tube 3 toward one end side of the ceiling plate 2 in the longitudinal direction.

Further, a rear stage of the FPD 4 is provided with the image generation unit 11 which generates the perspective image PO on the basis of a detection signal output therefrom (see FIG. 1) and a further rear stage of the image generation unit 11 is provided with the tomosynthesis image generation unit 12 which synthesizes the perspective image PO to generate the tomosynthesis image D. The image generation unit 11 corresponds to image generation means of the invention and the tomosynthesis image generation unit 12 corresponds to tomographic image generation means of the invention.

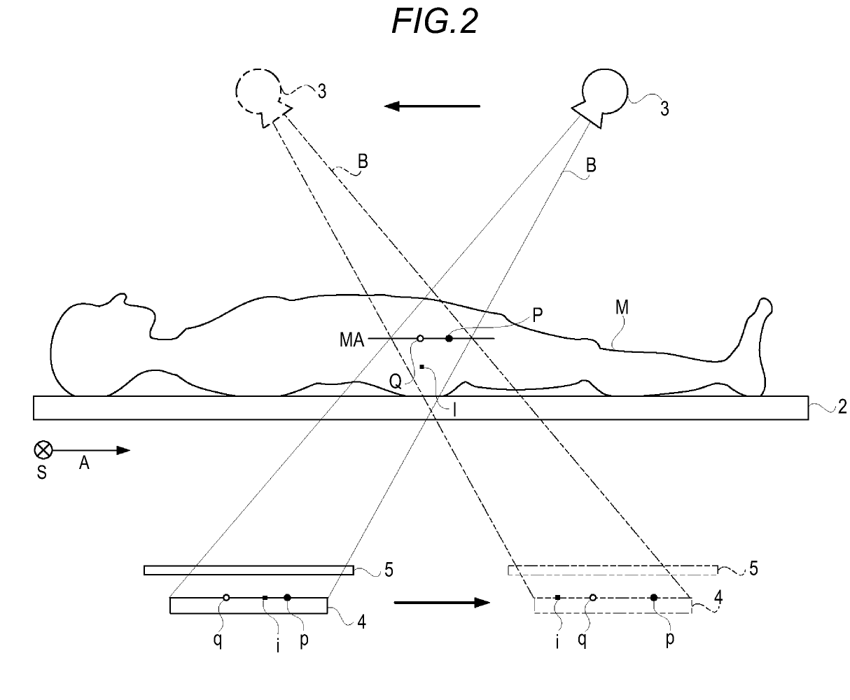

Next, a tomographic image acquiring principle of the bone analysis apparatus 1 according to the first embodiment will be described. FIG. 2 is a diagram showing a tomographic image acquiring method of an X-ray imaging apparatus according to the first embodiment. For example, a virtual plane (a standard cutting plane MA) which is parallel to the ceiling plate 2 (which is horizontal to the vertical direction) will be described. As shown in FIG. 2, a series of perspective images PO are generated by the image generation unit 11 while the FPD 4 is synchronously moved in a direction opposite to the X-ray tube 3 to match the irradiation direction of a cone-shaped X-ray beam B from the X-ray tube 3 so that points P and Q located on the standard cutting plane MA are respectively projected onto fixed points p and q of the X-ray detection surface of the FPD 4 at all times . Projected images of the subject M appear on the series of perspective images PO while the positions are changed. Then, when the series of perspective images PO are reconstructed by the tomosynthesis image generation unit 12, the images (for example, the fixed points p and q) located on the standard cutting plane MA are integrated and are imaged as an X-ray tomographic image. Meanwhile, a point I which is not located on the standard cutting plane MA appears as a point i on a series of subject images while the projection position of the FPD 4 is changed. Unlike the fixed points p and q, such a point i is blurred without forming an image at the stage of superimposing the X-ray transmission image by the tomosynthesis image generation unit 12. In this way, when the series of perspective images PO are superimposed, an X-ray tomographic image in which only the image located on the standard cutting plane MA of the subject M can be obtained. In this way, when the perspective images PO are simply superimposed, the tomosynthesis image D in which cross-sectional images of the subject M appear on the standard cutting plane MA can be obtained.

Further, the same tomographic image can be also obtained on an arbitrary cutting plane which is horizontal to the standard cutting plane MA by changing the setting of the tomosynthesis image generation unit 12. During the imaging, the projection position of the above-described point i of the FPD 4 changes, but the movement speed increases in accordance with an increase in separation distance between the standard cutting plane MA and the point I before projection. When the acquired series of subject images are reconstructed while being shifted at a predetermined pitch in the body axis direction A by using this fact, the tomosynthesis image D of the cutting plane parallel to the standard cutting plane MA can be obtained. The reconstruction of the series of subject images is performed by the tomosynthesis image generation unit 12. In this way, the tomosynthesis image generation unit 12 generates the tomosynthesis image D involving with a cross-section parallel to the ceiling plate for placing the subject M thereon on the basis of the continuously shot images while moving the X-ray tube 3 and the FPD 4 relative to the subject M.

Incidentally, the tomographic image of the subject M can be also obtained by an imaging method other than the above-described tomosynthesis imaging. However, the tomosynthesis imaging has a characteristic in which the tomographic image having a clear image of the trabecular bone appearing thereon is obtained compared to CT imaging which is one of other imaging methods. Thus, it can mention that the tomosynthesis imaging is an imaging method suitable for the trabecular analysis.

<Configuration of Image Analysis Unit>

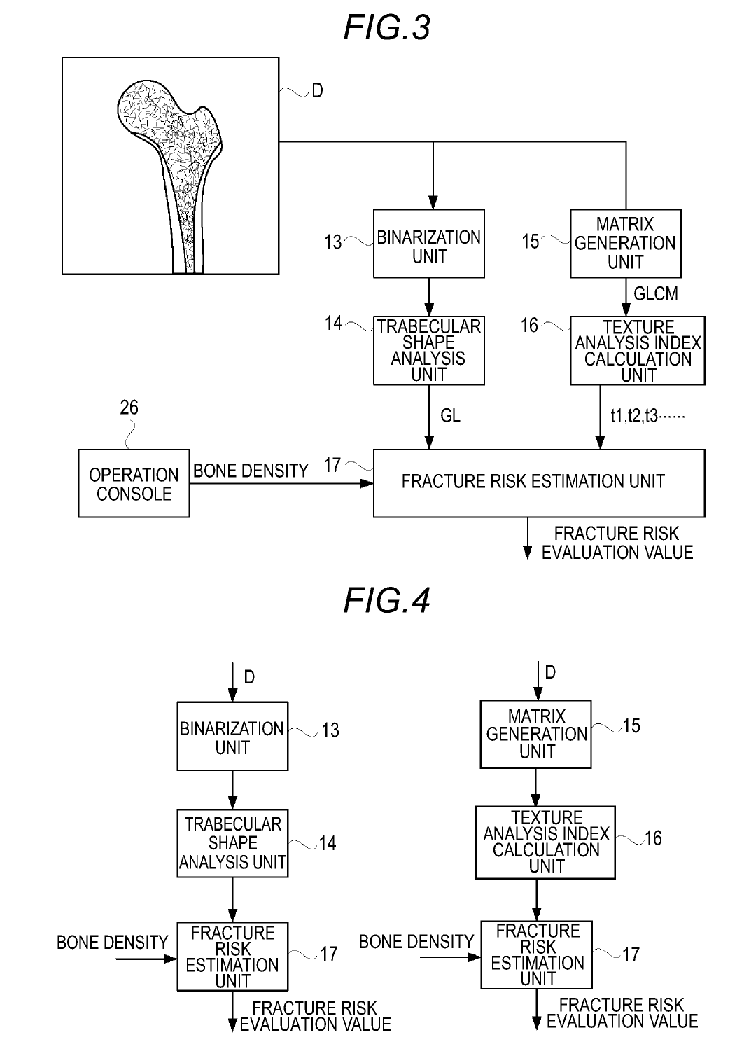

The generated tomosynthesis image D is transmitted to the image analysis units 13, 14, 15, 16, and 17. The image analysis units 13, 14, 15, 16, and 17 are expressed as one of functional blocks of the binarization unit 13, the trabecular shape analysis unit 14, the matrix generation unit 15, the texture analysis index calculation unit 16, and the fracture risk estimation unit 17 shown in FIG. 3. The image analysis units 13, 14, 15, 16, and 17 perform a bone analysis by performing various image processes on the tomosynthesis image D. The trabecular shape analysis unit 14, the matrix generation unit 15, the texture analysis index calculation unit 16 correspond to structural parameter calculation means of the invention and the fracture risk estimation unit 17 corresponds to fracture risk estimation means of the invention.

A configuration of the image analysis unit shown in FIG. 3 is an example of a configuration of the invention. As shown at the left side of FIG. 4, the image analysis unit may include the binarization unit 13, the trabecular shape analysis unit 14, and the fracture risk estimation unit 17. Then, as shown at the right side of FIG. 4, the image analysis unit may include the matrix generation unit 15, the texture analysis index calculation unit 16, and the fracture risk estimation unit 17.

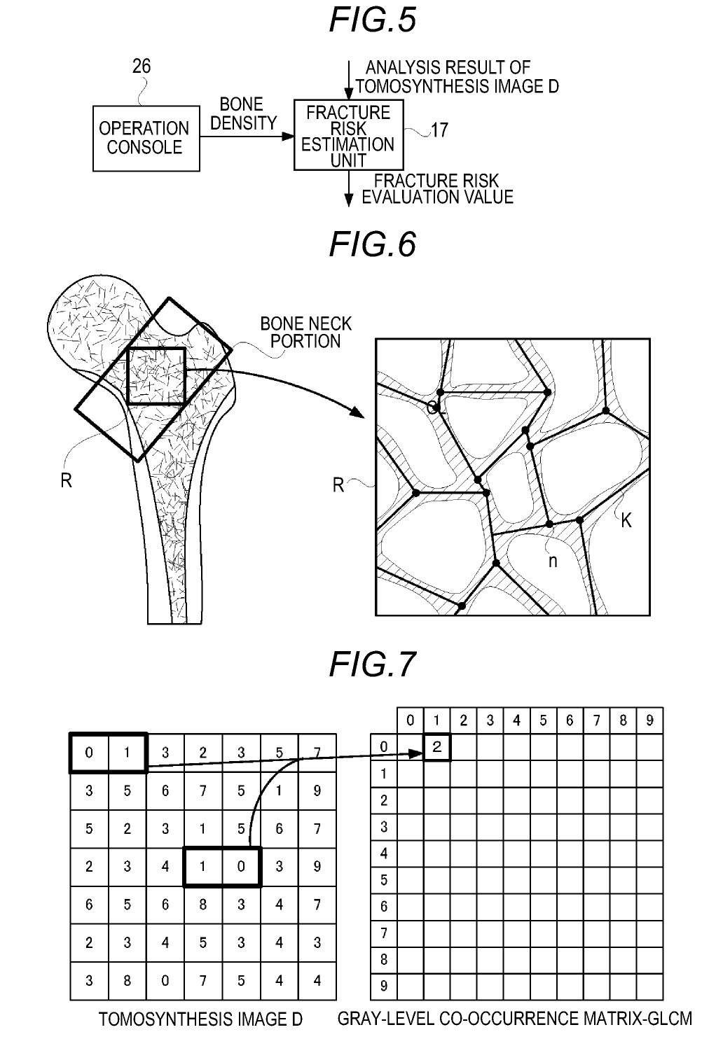

As shown in FIG. 5, the image analysis unit of the invention is configured to calculate the fracture risk evaluation value by adding a value indicating the bone density to the analysis result of the tomosynthesis image D. When any analysis is applied to the tomosynthesis image D, a structural parameter for evaluating a bone structure can be calculated by the analysis of the trabecular bone appearing on the tomosynthesis image D. All of the trabecular shape analysis unit 14, the matrix generation unit 15, and the texture analysis index calculation unit 16 are configured to calculate the structural parameter. At the time of analyzing the tomosynthesis image D, various structural parameters can be calculated when the analysis viewpoint is changed. The structural parameter to be used in the calculation of the fracture risk evaluation value by the fracture risk estimation unit 17 can be appropriately changed.

Thus, there is a case in which all structural parameters necessary for the fracture risk estimation unit 17 can be prepared by the trabecular shape analysis unit 14 or can be provided by the texture analysis index calculation unit 16. Further, there is a case in which all of the trabecular shape analysis unit 14 and the texture analysis index calculation unit 16 are necessary to calculate the structural parameter used by the fracture risk estimation unit 17. In the invention, a configuration including both of the trabecular shape analysis unit 14 and the texture analysis index calculation unit 16 will be described.

In this way, the image analysis unit of the invention is considered as various forms depending on the structural parameter used for analysis . However, in any form, it is common that the fracture risk estimation unit 17 uses the bone density in the calculation of the fracture risk evaluation value as shown in FIG. 5. The bone density is an index indicating a bone mineral content and is measured by an apparatus different from the apparatus shown in FIG. 1. The measurement of the bone density is performed by performing an imaging operation two times while changing the energy of the X-ray and analyzing the subtraction image which is a difference between two spot images. The subtraction image is an image obtained by capturing only the image of the bone of the subject M and unnecessary soft tissues are not reflected in the analysis. When the pixel value of the bone image appearing on the subtraction image is referred, the bone density can be accurately measured. The bone density is acquired on the basis of an inspection different from the imaging of the tomosynthesis image according to the bone analysis apparatus 1.

The bone density means the concentration of mineral (bone mineral or hydroxyapatite) with respect to the fastness of bone and is a numerical value showing a bone mineral content. Thus, the bone density is an important index for calculating the fracture risk evaluation value. Even though it is intuitive, it is easy to predict that the bone density will be higher when the fracture risk evaluation value is lower. The actual fracture risk evaluation value is almost the same as the expectation. Thus, it is a common sense in a medical field to measure the bone density when the fracture risk needs to know. However, the bone density does not indicate the fracture risk itself. That is, the inventor of the invention has found that the bone density alone is not enough to accurately calculate the fracture risk.

The inventor of the invention considers the influence of the bone structure as a reason why the fracture risk evaluation value cannot be accurately calculated only by the bone density. It is thought that the fracture risk evaluation value is different to some extent when the inner structure of the bone is different even in the same bone density. However, the conventional method of calculating the fracture risk evaluation value does not consider the bone structure at all. Thus, the conventional method cannot accurately measure the fracture risk evaluation value.

Here, the invention is made in consideration of the bone structure as well as the bone density at the time of calculating the fracture risk evaluation value. Thus, the invention can be easily understood when the analysis result (the structural parameter) of the tomosynthesis image D is also considered at the time of calculating the fracture risk evaluation value using the bone density. The bone structure mentioned herein specifically indicates the spongy bone structure of the subject M and the spongy structure composed of a plurality of trabecular bones insides the bones.

Next, the details of the image analysis units 13, 14, 15, 16, and 17 will be described.

<Binarization Unit 13 and Trabecular Shape Analysis Unit 14>

The tomosynthesis image D is first transmitted to the binarization unit 13. The binarization unit 13 generates a binarized tomosynthesis image D by performing a binarizing process on the tomosynthesis image D. The binarized tomosynthesis image D is transmitted to the trabecular shape analysis unit 14. The trabecular shape analysis unit 14 analyzes the trabecular bone appearing on the analysis range R provided in apart of the tomosynthesis image D and calculates the result. FIG. 4 is a schematic diagram showing an operation of the trabecular shape analysis unit 14. The left side of FIG. 6 shows the tomographic image of the bone of the subject M appearing on the tomosynthesis image D. The trabecular shape analysis unit 14 recognizes a part of a spongy material inside bone as an analysis range R.

A right side of FIG. 6 shows an enlarged view of the analysis range R. A plurality of tomographic images of trabecular bones appear in the analysis range R. The trabecular bone forms a meshed spongy material. The trabecular shape analysis unit 14 calculates various structural parameters by analyzing the trabecular bone image appearing in the analysis range R. The structural parameter numerically shows the characteristics of the spongy structure composed of the trabecular bone.

The trabecular shape analysis unit 14 calculates, for example, the structural parameters including the value BV/TV, the value TSL, the value TbTh, and the like by analyzing the analysis range R. These structural parameters numerically show the shape of the trabecular bone. The value BV/TV indicates a ratio between a part included in the trabecular bone of the analysis range R and the other part. The value BV/TV shows a volume ratio some times, but is understood as an area ratio inside the analysis range R of the invention.

The value BV/TV may be confused with the bone density, but both have different concepts. The bone density is a density of a bone obtained without considering the trabecular bone structure. The bone density is a numerical value showing how many contents of bone mineral (hydroxyapatite) are included in a specific region, that is, the density of bone mineral. The value BV/TV is a numerical value showing how many trabecular bones are included in a specific region, that is, a ratio between a space occupied by the trabecular bone and a space occupied by a gap.

The value TSL means the total extension of the trabecular bone appearing in the analysis range R. As shown in FIG. 6, the TSL can be obtained by acquiring branch points n of the trabecular bone in the analysis range R through the image analysis, acquiring lines K connecting the branch points n, and adding the lengths of the lines K.

The value TbTh means the thickness of the trabecular bone. The value TbTh can be obtained by the average value of the thickness of the trabecular bone included in the analysis range R. The number of the structural parameters calculated by the trabecular shape analysis unit 14 is not limited to three parameters described above.

In the configuration of the invention, the structural parameters can be also calculated by the texture analysis. The structural parameter is calculated from a viewpoint different from the analysis of the trabecular shape analysis unit 14. Even in this case, there is no change that the structural parameter is an evaluation value at the time of evaluating the trabecular bone structure. The texture analysis has a relation with the matrix generation unit 15 and the texture analysis index calculation unit 16.

<Matrix Generation Unit 15>

As a matrix necessary for the texture analysis, a gray-level co-occurrence matrix (GLCM) is known. The matrix is generated by the matrix generation unit 15. The tomosynthesis image D which is generated by the tomosynthesis image generation unit 12 is transmitted to the matrix generation unit 15 and is converted into the GLCM. FIG. 7 shows an operation of generating the GLCM on the basis of the tomosynthesis image D by the matrix generation unit 15. The left side of FIG. 7 shows the tomosynthesis image D as a two-dimensional array of the pixel value. For the simple description, it is assumed that each pixel value of the pixels constituting the tomosynthesis image D takes ten values from zero to nine.

As shown at the right side of FIG. 7, the number of rows and columns of the GLCM generated from the tomosynthesis image D matches the number of the pixel values taken as the pixel values of pixels. Since each of the pixels constituting the tomosynthesis image D has any pixel value from ten values, the GLCM generated from the tomosynthesis image D becomes a two-dimensional matrix of ten rows and ten columns. The matrix generation unit 15 completes the GLCM by applying numerical values to one hundred elements constituting the GLCM corresponding to the 10×10 matrix. The numerical value to be applied to each element is determined on the basis of the pixel value of the tomosynthesis image D.

FIG. 7 shows a state where the matrix generation unit 15 determines a numerical value of an element p (0, 1) located at a row corresponding to zero in each row and a row corresponding to one in each column in the GLCM. The matrix generation unit 15 counts how many pairs of pixels being adjacent to each other and having a pixel value of zero and a pixel value of one are arranged in the tomosynthesis image D and sets the count number as the element p (0, 1) of the GLCM. In FIG. 7, since there are two pairs of pixels of which the pixel value 0 and the pixel value 1 are adjacent to each other, the value of the element p (0, 1) becomes two. Since an arbitrary element p (a, b) in the GLCM is the same as the element p (b, a), the value of the element p (1, 0) of the GLCM is also two.

The matrix generation unit 15 determines all elements of the matrix on the basis of the tomosynthesis image D by performing the same operation in the entire GLCM. In this way, the matrix generation unit 15 completes the GLCM on the basis of the tomosynthesis image D.

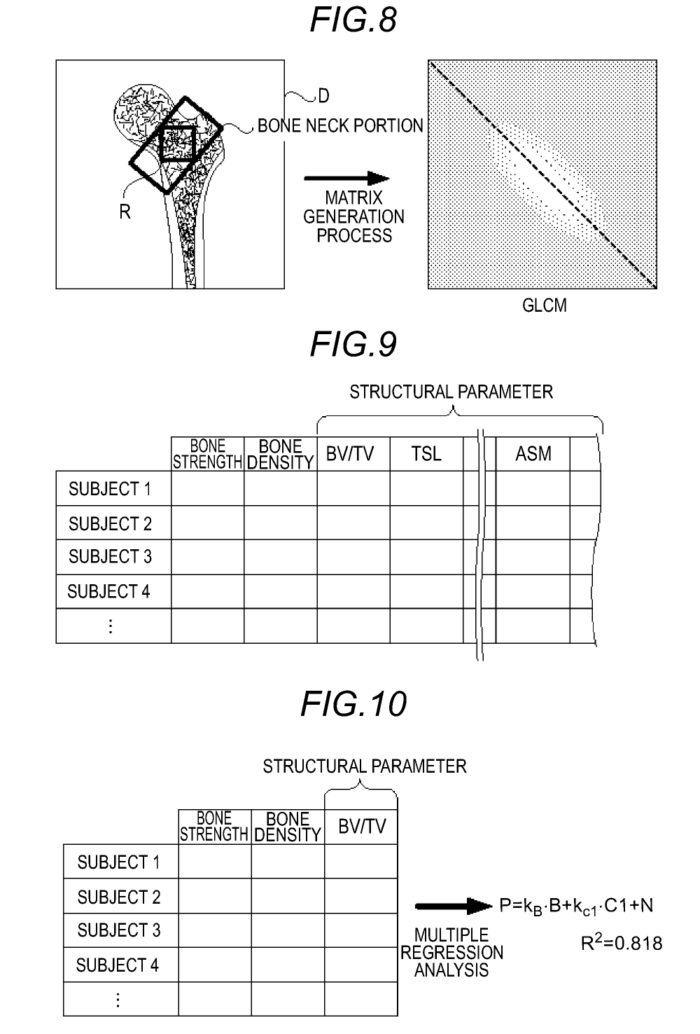

FIG. 8 shows a state where the matrix generation unit 15 generates the GLCM on the basis of the tomosynthesis image D. The generated GLCM increases as the number of the pixel values taken by the pixels of the tomosynthesis image D increases. The GLCM is a matrix having symmetry and is a matrix in which the values of overlapping elements are the same when the matrix is folded in half with a diagonal line indicated by a dotted line in FIG. 8.

In this way, the matrix generation unit 15 generates the GLCM (the gray-level co-occurrence matrix) by counting the number of the pixels separated from each other by a predetermined distance and appearing in the analysis range as a combination of the pixel values on the assumption that a pair of two pixels have a combination of predetermined pixel values among the pixels constituting the analysis range provided in a part of the tomosynthesis image D. The matrix generation unit 15 generates the GLCM for the spongy bone of each part of the bone appearing on the tomosynthesis image D. Specifically, each part of the bone is a bone neck portion or a bone stem portion. FIG. 8 shows a state where the GLCM for the bone neck portion is generated.

<Texture Analysis Index Calculation Unit 16>

The GLCM is transmitted to the texture analysis index calculation unit 16. The texture analysis index calculation unit 16 can calculate a texture analysis index by performing various calculations on the GLCM. The texture analysis index which can be calculated by the texture analysis index calculation unit 16 is, for example, as below. p (i, j) in the expression indicates a value of the element at the i-th row and the j-th column in the GLCM, Σᵢ and Σⱼ indicate the sum of the elements at the i-th row and the j-th column, N_{g} indicates the number of the pixel values taken by the pixels of the tomosynthesis image D, μ indicates an average value, μₓ and μ_{y} respectively indicate average values in the row direction and the column direction, and σₓ and σ_{y} respectively indicate standard deviations in the row direction and the column direction. In addition, the texture analysis indexes ASM (Angular Second Moment), CNT (Contrast), COR (Correlation), VAR (Variance), IDM (Inverse Difference Moment), and ENT (Entropy) are a part of fourteen kinds of parameters proposed in the following document (A) by Harlick and the like in 1973. Further, DIS is the texture analysis index called non-similarity or dissimilarity and HOM is the texture analysis index called uniformity or homogeneity.

(A) Haralick RM. et al. Textural Features for Image Classification. IEEE Transactions on Systems Man and Cybernetics 1973; 6:610-621.

### Expression 1

The texture analysis index calculation unit 16 calculates the texture analysis index by performing the above-described various calculations on the GLCM. The number and the type of the texture index calculated by the texture analysis index calculation unit 16 can be appropriately changed. The number of the texture analysis indexes may be three or less. As described above, the texture analysis index calculation unit 16 calculates the texture analysis index by performing the texture analysis on the basis of the GLCM (Gray-Level Co-occurrence Matrix). The texture analysis index is a kind of the structural parameter of the invention.

As described above, the trabecular shape analysis unit 14 and the texture analysis index calculation unit 16 calculate the structural parameter on the basis of the tomosynthesis image of the subject M. Various structural parameters calculated in this way are transmitted to the fracture risk estimation unit 17. The fracture risk estimation unit 17 calculates the fracture risk evaluation value on the basis of the estimation expression when a predetermined structural parameter is input thereto. In order to calculate the fracture risk evaluation value by the fracture risk estimation unit 17, the bone density is necessary in addition to the above-described structural parameter. The bone density is measured in advance by the subtraction imaging before the imaging of the tomosynthesis image D according to the structural parameter. The operator can input the bone density through the operation console 26. In this way, the fracture risk estimation unit 17 of the invention is configured to calculate the fracture risk evaluation value indicating a risk of causing the fracture of the bone in consideration of the bone density indicating the density of the material relating to the fastness of the bone of the subject M and the structural parameter for evaluating the structure of the trabecular bone forming the bone of the subject M.

The fracture risk estimation unit 17 calculates the fracture risk evaluation value by applying the structural parameter corresponding to the analysis result of the tomosynthesis image D and the bone density input through the operation console 26 by the operator to the estimation expression. At this time, the estimation expression used in the calculation by the fracture risk estimation unit 17 is, for example, as below. As the fracture risk evaluation value decreases, the risk of the fracture exists.

P=k_{B}·B+k_{C}·C+N (1)

Here, P indicates the fracture risk evaluation value, B indicates the bone density, C indicates the structural parameter, and N indicates a constant. k_{B} and k_{C} are coefficients multiplied by each parameter. As the structural parameter, the value BV/TV and the like which are calculated by the trabecular shape analysis unit 14 may be used or the ASM and the like which are calculated by the texture analysis index calculation unit 16 may be used. Further, the estimation expression may include two or more structural parameters, for example, as below.

P=kB·_{B}+k_{C1}·C1+k_{C2}·C2+ . . . +N

In this way, in the invention, it is possible to appropriately select the kind and the number of the structural parameters of the estimation expression. A common point of the estimation expression of the invention is that the estimation expression includes a term involving with the bone density and a term involving with the structural parameter. That is, the fracture risk estimation unit 17 is configured to calculate the fracture risk evaluation value by using the estimation expression showing a correlation among the fracture risk evaluation value, the bone density, and the structural parameter.

<Determination of Estimation Expression>

A method of determining the estimation expression used in the operation of the fracture risk estimation unit 17 will be described. In order to complete the estimation expression, it is necessary to determine a certain parameter to be used among several structural parameters and to determine a coefficient and a constant for each part of the bone. Such an estimation expression can be determined before the trabecular analysis of the subject M. As a method of determining the estimation expression, a method of using a regression can be used.

First, the fracture risk evaluation value, the bone density, and the structural parameter are calculated by actually analyzing the subject for the plurality of subjects M.

The fracture risk evaluation value of the subject is obtained by a simulation of CT Finit Element Method (FEM). This method is to acquire a 3D image of a spongy bone by CT imaging and to generate a three-dimensional model on the basis of the image. Then, a simulation is performed by assuming a case where a physical load is applied to the three-dimensional model and it is estimated how well this structure can endure a force without breakage. The estimation value indicating the estimation result is the fracture risk evaluation value. The fracture risk evaluation value can be measured by such a method, but since the method is complex and requires a complex calculation, it is difficult to mention that this method is easy for the examination of the subject M.

The bone density of the femoral neck portion can be obtained by capturing the subtraction image of the femur as described above. The bone density means the density of the bone mineral realizing the strength of the bone. Further, the structural parameter of the femoral neck portion can be obtained by capturing the tomosynthesis image as described above. The structural parameter is an evaluation value for evaluating the state of the trabecular bone. FIG. 9 shows a table in which various parameters obtained in this way are arranged for each subject M.

Next, the estimation expression is finally determined. According to the configuration of the invention, a plurality of estimation expressions having different structural parameters are provided and an estimation expression capable of most accurately estimating the fracture risk evaluation value is selected. As an example, an estimation expression using the value BV/TV as the structural parameter and an estimation expression using the value TSL as the structural parameter are obtained and the better estimation expression is determined. First, a multiple regression analysis on the value BV/TV is performed. The multiple regression analysis is a statistical method of calculating an expression that predicts one parameter from a plurality of parameters. The multiple regression analysis is to determine two parameters and one parameter involving with the parameters and to perform a statistical analysis to obtain an estimation expression. When two parameters are input to the estimation expression, one parameter is output. Two parameters involving with the input will be referred to as independent variables and a parameter involving with the output will be referred to as an dependent variable.

In order to understand the method of calculating the estimation expression for the value BV/TV, a table shown in FIG. 10 is helpful. FIG. 10 only shows the fracture risk evaluation value, the bone density, and the value BV/TV from the table shown in FIG. 9. When the estimation expression for the value BV/TV is calculated, the independent variables are the bone density and the value BV/TV and the dependent variable is the fracture risk evaluation value. When the multiple regression analysis is performed by using such a data group, an expression shown in (1) and a value R² indicating the estimation reliability are calculated. In general, it is understood that the reliability of the estimation expression is high as the value R² is close to one. When the reliability of the estimation expression is high, it means that a difference in numerical value between the actual data and the estimation result using the estimation expression is small.

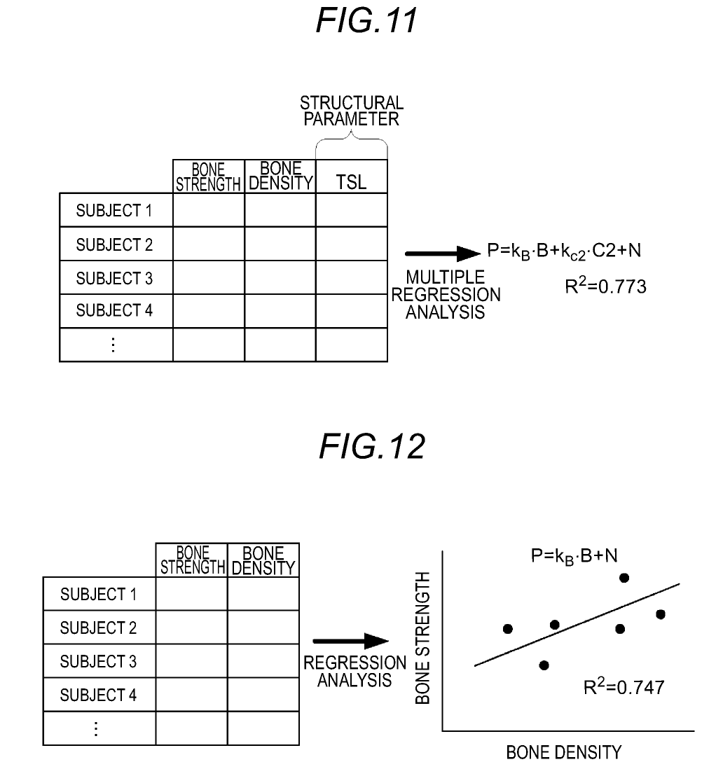

FIG. 11 shows a state where the estimation expression for the value TSL is calculated. FIG. 11 only shows the fracture risk evaluation value, the bone density, and the value TSL from the table shown in FIG. 9. When the estimation expression for the value TSL is calculated, the independent variables are the bone density and the value TSL and the dependent variable is the fracture risk evaluation value. When the multiple regression analysis is performed by using such a data group, an expression shown in (1) and a value R² indicating the estimation reliability are also calculated.

In this way, the value R² is an original value which indicates the reliability of each estimation expression. When the values R² between the estimation expressions are compared, it is possible to determine an estimation expression suitable for the calculation of the fracture risk evaluation value. With reference to the examples of FIGS. 10 and 11, the estimation expression suitable for the calculation of the fracture risk evaluation value is the estimation expression involving with the value BV/TV. This is because the value R² involving with the value BV/TV is larger than the value R² involving with the value TSL.

The estimation expression of the invention is selected so that the fracture risk evaluation value is most accurately estimated from the plurality of estimation expressions having different structural parameters on the basis of such a principle. In the examples shown in FIGS. 10 and 11, the estimation expression is calculated by performing the multiple regression analysis using one structural parameter other than the bone density as an independent variable, but the estimation expression may be calculated by performing the multiple regression analysis using a plurality of structural parameters other than the bone density as independent variables.

<Effect of Invention>

Finally, since the effect of the invention has been proved, this effect will be described. That is, as a demonstration, the fracture risk evaluation value, the bone density, and the structural parameters of the femoral neck portion were actually calculated from in thirty nine diabetic patients. The reason why the diabetic patients are used to prove the effect of the invention is that it is considered that the fracture risk evaluation value of a diabetic patient is not easily and accurately calculated only by the bone density, so that this case more noticeably shows the effect of the invention.

FIG. 12 shows a method of calculating the fracture risk evaluation value only by the bone density as in the related art. That is, the estimation expression of estimating the fracture risk evaluation value by the bone density was calculated by performing the regression analysis using the bone density and the fracture risk evaluation value measured for each subject M. A value R² obtained at this time was 0.747. The estimation expression obtained at this time is as below like the expression (1).

P=k_{B}·B+N

Next, as shown in FIG. 10, a method of calculating the fracture risk evaluation value by the bone density and the value BV/TV corresponding to a kind of structural parameter was performed. That is, the estimation expression of estimating the fracture risk evaluation value by the bone density and the value BV/TV was calculated by performing the regression analysis on the bone density, the value BV/TV, and the fracture risk evaluation value measured for each subject M. The estimation expression obtained at this time is as below. This expression is similar to Expression (1) described above.

P=10,759×B+11,430×C−3,278 (2)

A value R² of the estimation expression was 0.818. The estimation value is higher than the value R² of the estimation expression of obtaining the fracture risk evaluation value by performing the regression analysis only using the bone density. Thus, a more highly reliable result was obtained when calculating the fracture risk evaluation value using the bone density and the value BV/TV.

At the time of examining the subject M, it is difficult to calculate the fracture risk evaluation value by the CT Finit Element Method. However, when the fracture risk evaluation value is obtained by using the estimation expression shown in Expression (2) described above, the fracture risk evaluation value can be simply calculated just by calculating the bone density and the structural parameter which are relatively easily measured. Further, the reliability of the calculated fracture risk evaluation value is high in Expression (2) as indicated by the value R² thereof.

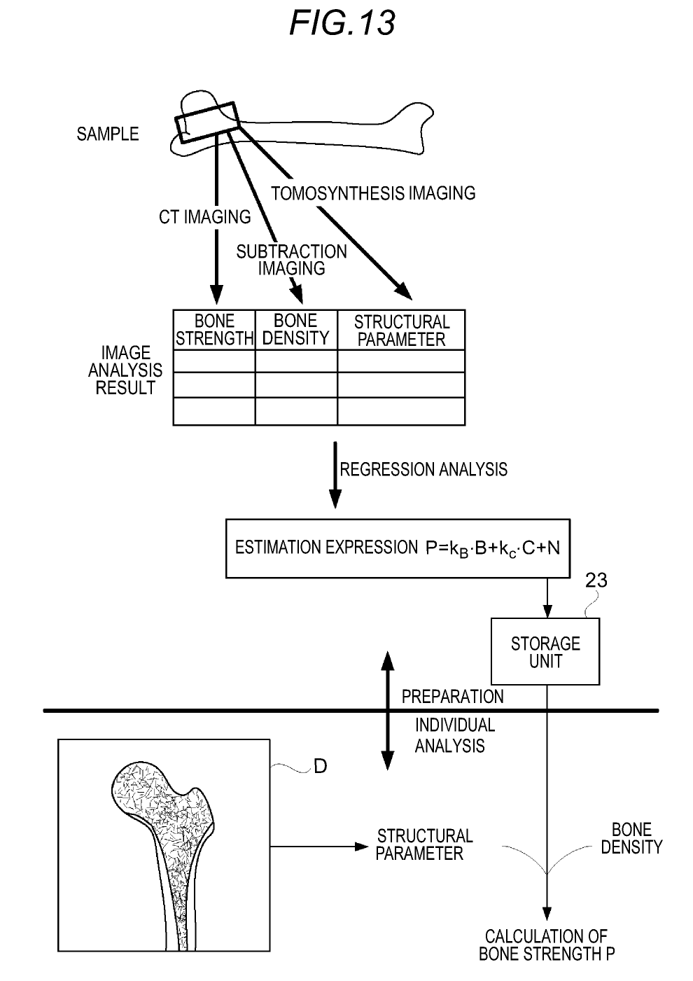

FIG. 13 is a summary of the invention. As a preparation of the bone analysis according to the invention, an image analysis is first performed on each of images obtained by CT imaging, subtraction imaging, and tomosynthesis imaging on samples. An estimation expression is calculated by performing a multiple regression analysis using the bone density and the structural parameter as independent variables and using the fracture risk evaluation value as a dependent variable among the image analysis results. The estimation expression has the highest reliability (highest value R²) among the estimation expressions calculated by changing the independent variables. A case of FIG. 13 shows a state where the estimation expression for the bone neck portion is calculated.

The estimation expression which is prepared in advance is stored in the storage unit 23. At the time of performing the bone analysis of the subject M, the subtraction imaging is first performed in advance to calculate the bone density. The bone density is input to the bone analysis apparatus 1 through the operation console 26 by the operator. Then, tomosynthesis imaging is performed by using the bone analysis apparatus 1. The operator calculates the structural parameter corresponding to the independent variable of the estimation expression on the basis of the tomosynthesis image D in which the bone density is input to the bone analysis apparatus 1 through the operation console 26. The fracture risk estimation unit 17 calculates the fracture risk evaluation value indicating the strength of the bone on the basis of the calculated structural parameter, the input bone density, and the estimation expression stored in the storage unit 23.

As described above, according to the invention, it is possible to calculate a more highly reliable result in the bone analysis apparatus which calculates the fracture risk evaluation value on the basis of the bone density. That is, the bone density of the invention is positioned for a partial description of the fracture risk. That is, in the invention, it is considered that an accurate fracture risk cannot be sufficiently obtained only by the bone density although the bone density is important to know the fracture risk. The same applies to the structural parameter. That is, in the invention, it is considered that an accurate fracture risk cannot be sufficiently obtained only by the structural parameter although the structural parameter is important to know the fracture risk. According to the invention, the fracture risk is comprehensively evaluated on the basis of the structural parameter for evaluating a structure of a trabecular bone in addition to the bone density. With this configuration, since the fracture risk can be evaluated from two viewpoints of the bone density and the structure of the trabecular bone, the fracture risk can be more accurately evaluated.

Further, since the structural parameter can be calculated on the basis of an image in which the trabecular bone clearly appears when the structural parameter is calculated on the basis of the tomosynthesis image of the subject M as described above, the fracture risk can be more accurately evaluated.

<Effect of Invention>

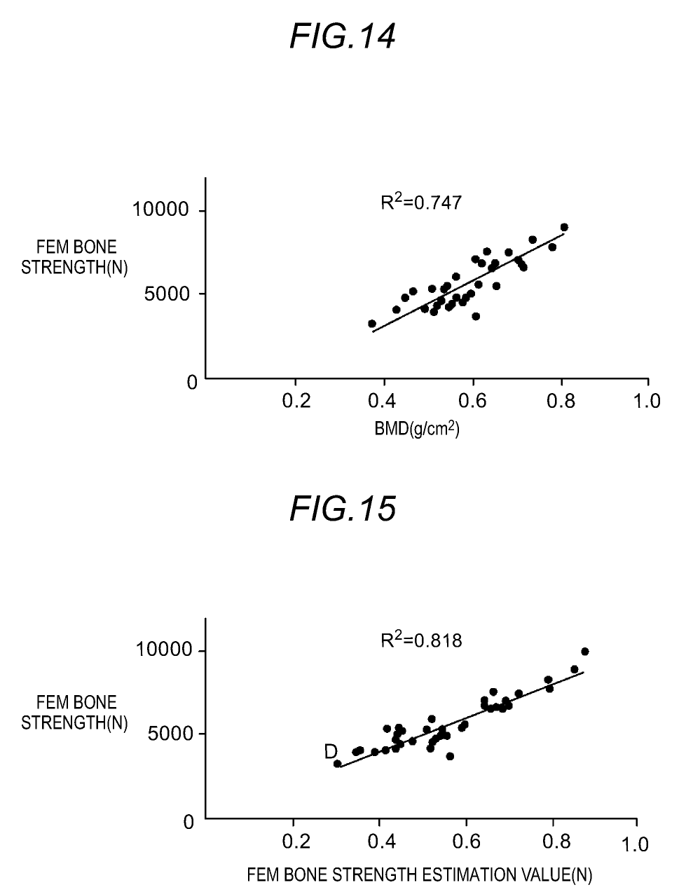

FIGS. 14 and 15 illustrate an effect of the invention. FIG. 14 shows a correlation between the actual bone strength and the bone density calculated by the X-ray imaging analysis. In the conventional configuration, the bone density indicates the bone strength. That is, it is a premise that there is a correlation between the bone strength and the bone density calculated by the X-ray image analysis. FIG. 14 shows the degree of the correctness of the premise. Here, a bone density (BMD) obtained by an image analysis on a certain part of a sample bone and a bone strength measured by applying a pressure thereto are plotted as a result. Thus, a FEM bone strength of the vertical axis is not obtained from the measurement of the actual subject. Referring to FIG. 14, it is understood that there is a positive correlation between the bone density and the bone strength as an entire tendency. However, the result is slightly scattered, and the value R² obtained by the regression analysis is 0.747.

FIG. 15 shows a plot obtained by the bone analysis apparatus according to the invention. An FEM bone strength estimation value (N) of the horizontal axis is obtained by calculating a neck BMD value of the sample bone and the value BV/TV corresponding to the structural parameter for each part of the sample bone and estimating the FEM bone strength of each part of the sample bone on the basis of the expression obtained by the regression analysis performed on the obtained result. At this time, in the estimation expression described in FIG. 13, k_{B} was 10,759, k_{C} was 11,430, and N was −3,278. Additionally, the estimation expression is obtained by the bone strength measurement and the image analysis of the bone neck portion.

FIG. 15 shows a plot of the result for the FEM bone strength estimation value obtained by the image analysis on the bone neck portion of the sample bone and the measured bone strength obtained by applying a pressure thereto. Thus, an FEM bone strength of the vertical axis is not obtained by the measurement of the actual subject. As an entire tendency, it is understood that there is a correlation between the bone density and the bone strength. A value R² obtained by the regression analysis was 0.818.

When a result corresponding to the conventional configuration of FIG. 14 is compared with the configuration according to the invention of FIG. 15, it is proved that the value R² according to the invention is higher than the value R² according to the related art. That is, a method of calculating the fracture risk according to the invention can accurately calculate the fracture risk compared to the related art.

### Second Embodiment

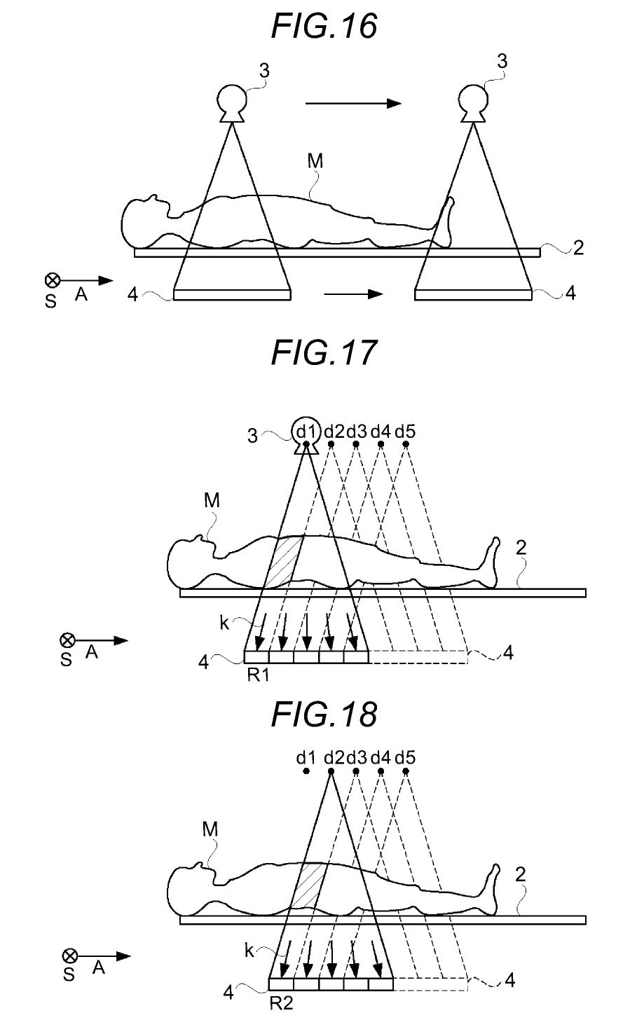

Next, a bone analysis apparatus according to a second embodiment will be described. A configuration of the second embodiment is a configuration in which the X-ray tube 3 and the FPD 4 capture the tomographic image while moving in the body axis direction A of the subject M in a state of keeping a relative positional relation as shown in FIG. 16. That is, the synchronous movement mechanism 7 moves the FPD 4 toward one end of the ceiling plate 2 in the longitudinal direction in synchronization with the movement of the X-ray tube 3 toward one end of the ceiling plate 2 in the longitudinal direction.

A configuration of the X-ray imaging apparatus according to the second embodiment is similar to the functional block diagram of FIG. 1. The configuration of the second embodiment is different from that of the first embodiment in FIG. 1 in that the FPD 4 moves following the X-ray tube 3 (see FIG. 16) and the X-ray tube 3 is not inclined. Thus, in the second embodiment, the X-ray tube tilt mechanism 9 and the X-ray tube tilt control unit 10 of FIG. 1 are not essentially required.

A tomographic image capturing principle according to the second embodiment will be described. First, as shown in FIG. 16, the imaging systems 3 and 4 intermittently irradiate X-rays while moving relative to the subject M in the state of keeping a relative position. That is, whenever each irradiation ends, the X-ray tube 3 moves in the body axis direction A of the subject M and irradiates the X-rays again. In this way, a plurality of transmission images are acquired and a processed image (an elongated transmission image to be described later) of the transmission images is reconstructed into a tomographic image by a filter back projection method. The completed tomographic image is an image with a tomographic image obtained by cutting the subject M along a certain cutting plane.

In order to generate the tomographic image, an image when the subject M is seen through from different directions is required. The bone analysis apparatus according to the second embodiment divides the obtained transmission image and joins the divided images to generate the tomographic images. This operation will be described. FIG. 17 shows a position of the FPD 4 when a focal point of the X-ray of the X-ray tube 3 is located at the position of d1. In this imaging, it is assumed that the transmission image is captured when the X-ray tube 3 and the FPD 4 move in the body axis direction A of the subject M relative to the ceiling plate 2 by the width of ⅕ of the FPD 4 in this direction.

The X-ray is radially widened from the X-ray tube 3 to reach the FPD 4. For this reason, when the generated transmission image is divided into five segments in the body axis direction A of the subject M, the incident angle of the X-ray with respect to the FPD 4 is different among the segments as indicated by an arrow. A direction k of one of these segments will be mainly described. Since the X-ray traveling in the direction k passes through a hatched part of the subject M and appears on the FPD 4, the hatched part of the subject M appears in the segments of the FPD 4 to which the X-ray is incident in the direction k. In the transmission image, a part corresponding to this segment will be referred to as a fragment R1.

FIG. 18 shows a position of the FPD 4 when a focal point of the X-ray of the X-ray tube 3 is located at the position of d2 moved from the position dl by the width of ⅕ of the FPD 4. Since a positional relation between the X-ray tube 3 and the FPD 4 does not change, the FPD 4 has a segment to which the X-ray traveling in the direction k appears even in this imaging and a hatched part of the subject M appears in the segment of the FPD 4 to which the X-ray traveling in the direction k is incident. In the transmission image, a part corresponding to the segment will be referred to as a fragment R2.

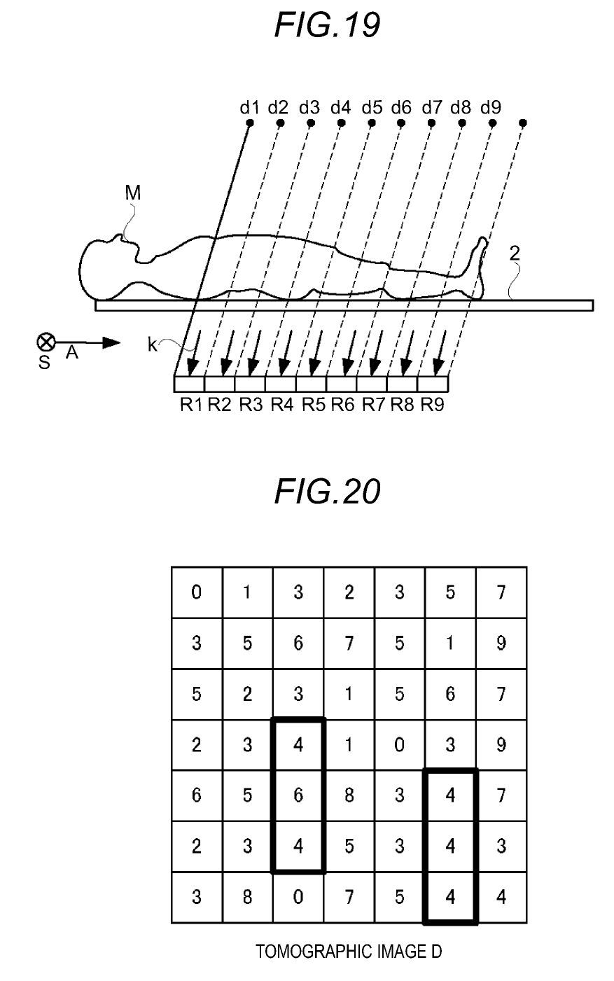

Since the positions of the subject M relative to the imaging systems 3 and 4 are different when the fragment R1 and the fragment R2 are compared with each other, the subject M appearing in both fragments R1 and R2 are different from each other. Here, the X-ray tube 3 is offset by the width of ⅕ of the FPD 4. For this reason, when the imaging is performed nine times at focal points d1 to d9, different positions of the subject M appear in fragments R1 to R9 of the transmission image in the segments of the FPD 4 to which the X-ray is incident in the direction k at this time. Here, as shown in FIG. 19, when the fragments R1 to R9 of the transmission image are connected in this order in the body axis direction A of the subject M, it is possible to obtain an image captured when the X-ray is irradiated to the whole body of the subject M in a certain direction k. This image will be referred to as an elongated transmission image.

The bone analysis apparatus according to the second embodiment generates an elongated transmission image also in a direction other than the direction k in the tomosynthesis image generation unit 12. Then, the tomosynthesis image generation unit 12 is used to generate the tomosynthesis image D when the subject M is cut at a predetermined cutting position based on a plurality of elongated transmission images having different projection directions of the subject M.

The analysis of the tomosynthesis image D in the second embodiment is similar to that of the first embodiment and the fracture risk evaluation value is finally calculated.

As described above, according to the configuration of the second embodiment, the tomosynthesis image D is captured from a long image acquired by virtually performing slot photographing. When such imaging is performed, it is possible to provide a radiation imaging apparatus capable of acquiring the tomosynthesis image D captured in a broad range.

The invention is not limited to the above-described configuration and can be modified as below.

(1) In the above-described embodiment, the texture analysis index is shown, but the fracture risk evaluation value can be calculated by using other texture analysis indexes derived from the gray-level co-occurrence matrix. That is, the texture analysis indexes other than those proposed by Harlick and the like exemplified in the above-described embodiments can be used.

(2) In the above-described configuration, the fracture risk evaluation value is expressed as a continuous numerical value, but the invention is not limited to this configuration. The fracture risk estimation unit 17 may express whether the fracture risk is high or low by two values. In the case of such a configuration, the fracture risk evaluation value means a flag for identifying the fracture risk. In addition, the fracture risk evaluation value may be determined so that the fracture risk is evaluated by a predetermined step.

(3) In the above-described configuration, the bone density is input by the operator, but the invention is not limited to this configuration. The fracture risk estimation unit 17 may be configured to read the bone density stored in the storage unit 23.

(4) The matrix generation unit 15 of the above-described embodiment is configured to count the number of the pairs of adjacent pixels in the tomosynthesis image D, but the invention is not limited to this configuration. That is, as shown in FIG. 18, the gray-level co-occurrence matrix may be generated by counting the number of the pairs of the pixels which are separated from each other by a predetermined distance. An example of FIG. 20 shows a state where the matrix generation unit 15 counts the number of the pairs of the pixels which are separated from each other by the width of one pixel and of which both pixel values are four.

(5) In the above-described embodiment, the trabecular shape analysis unit 14 is configured to calculate the structural parameter such as the value BV/TV, but the invention is not limited to this configuration. In the invention, the trabecular shape analysis unit may be configured to calculate the structural parameter involving with the evaluation of the trabecular bone like a trabecular number and an anisotropy and the fracture risk estimation unit 17 may be configured to calculate the fracture risk on the basis of the structural parameter.

(6) The multiple regression analysis of the above-described embodiment is performed according to the first order approximation method, but the invention is not limited to this configuration. The multiple regression analysis may be performed according to a second order approximation method. Further, the multiple regression analysis may be performed by a higher order approximation method.

(7) In the above-described embodiment, data showing a correlation among the fracture risk evaluation value, the bone density, and the structural parameter is expressed as an expression, but the invention is not limited to this configuration. The data showing the correlation may be provided as a database in which the parameters are managed as a table. Such a database can be obtained by the measurement or simulation of the parameters. When the data showing the correlation is provided as the database, the fracture risk estimation unit 17 recognizes a combination of the input bone density and the structural parameter and searches for the combination from the database to acquire the fracture risk evaluation value corresponding to the combination.

(8) According to the above-described embodiment, the structural parameter is calculated by the imaging result of the tomosynthesis apparatus, but the invention is not limited to the configuration. The structural parameter may be calculated by the imaging result of the CT apparatus other than the tomosynthesis apparatus.

(9) According to the above-described embodiment, the bone density is calculated by the subtraction imaging, but the invention is not limited to the above-described configuration. The bone density maybe calculated on the basis of the imaging result of the tomosynthesis apparatus.

### INDUSTRIAL APPLICABILITY

As described above, the invention is suitable for a medical field.

### REFERENCE SIGNS LIST

3 X-ray tube (radiation source)
4 FPD (detection means)
7a X-ray tube movement mechanism (radiation source movement means)
7b FPD movement mechanism (detector movement means)
8a X-ray tube movement control unit (radiation source movement control means)
8b FPD movement control unit (detector movement control means)
11 image generation unit (image generation means)
12 tomosynthesis image generation unit (tomographic image generation means)
14 trabecular shape analysis unit (structural parameter calculation means)
15 matrix generation unit (structural parameter calculation means)
16 texture analysis index calculation unit (structural parameter calculation means)
17 fracture risk estimation unit (fracture risk estimation means)
storage unit (storage means)
operation console (input means)

1-10. (canceled)

11. A bone analysis apparatus comprising:
fracture risk estimation means for calculating a fracture risk evaluation value for a subject by obtaining an estimation expression estimating the fracture risk evaluation value by a bone density and a structural parameter numerically showing characteristics of a spongy structure comprising a trabecular bone on the basis of the fracture risk evaluation value indicating a risk causing a fracture, the bone density, and the structural parameter, and inputting the bone density and the structural parameter obtained from the subject to the estimation expression.

12. The bone analysis apparatus according to claim 11,
wherein the fracture risk estimation means calculates the fracture risk evaluation value by using data showing a correlation among the fracture risk evaluation value, the bone density, and the structural parameter.

13. The bone analysis apparatus according to claim 11, further comprising:
structural parameter calculation means for calculating the structural parameter on the basis of a tomosynthesis image of the subject.

14. The bone analysis apparatus according to claim 13,
wherein the bone density is acquired on the basis of an inspection different from the capturing of the tomosynthesis image.

15. The bone analysis apparatus according to claim 11, further comprising:
input means for inputting the bone density by an operator.

16. The bone analysis apparatus according to claim 11, further comprising:
storage means for storing the bone density.

17. The bone analysis apparatus according to claim 13,
wherein the structural parameter calculation means calculates any one of a value BV/TV indicating a ratio between a bone component inside an interested region involved with the calculation of the structural parameter and the other part, a value TSL indicating a total extension of the trabecular bone, and a value TbTh indicating a width of the trabecular bone as the structural parameter.

18. The bone analysis apparatus according to claim 13, further comprising:
gray-level co-occurrence matrix generation means corresponding to the structural parameter calculation means and generating a gray-level co-occurrence matrix by counting a number of times pixels separated from each other by a predetermined distance and appearing in an interested region as a combination of pixel values, a pair of two pixels having a combination of predetermined pixel values among pixels constituting the interested region involved with the calculation of the structural parameter; and
texture analysis means for performing a texture analysis on the basis of the gray-level co-occurrence matrix and calculating a texture analysis index corresponding to the structural parameter as the structural parameter.

19. The bone analysis apparatus according to claim 18,
wherein one or more of correlation, dissimilarity, contrast, homogeneity, entropy, angular second moment, variance, and inverse differential moment are selected as the texture analysis index calculated by the texture analysis means.

20. The bone analysis apparatus according to claim 13, further comprising:
a radiation source that irradiates a radiation;
radiation source movement means for moving the radiation source relative to the subject;
radiation source movement control means for controlling the radiation source movement means;
detection means for detecting radiation transmitted through the subject;
detector movement means for moving the detection means relative to the subject;
detector movement control means for controlling the detector movement means;
image generation means for generating an image on the basis of an output of the detection means; and
tomographic image generation means for generating the tomosynthesis image on the basis of a continuous shot image obtained while moving the radiation source and the detection means relative to the subject.

21. The bone analysis apparatus according to claim 11,
wherein the fracture risk evaluation value, the bone density, and the structural parameter used when the fracture risk estimation means obtains the estimation expression are calculated by analyzing a plurality of subjects.

22. The bone analysis apparatus according to claim 11,
wherein the fracture risk evaluation value, the bone density, and the structural parameter used when the fracture risk estimation means obtains the estimation expression are obtained by X-ray imaging for a sample bone.
